(19) United States
(12) Patent Application Publication
(10) Pub. No.: US 2009/0118600 A1
(43) Pub. Date: May 7, 2009

(54) METHOD AND APPARATUS FOR SKIN DOCUMENTATION AND ANALYSIS

(76) Inventors: Joseph L. Ortiz, Knoxville, TN (US); Nancy A. Ortiz, Knoxville, TN (US)

Correspondence Address: LUEDEKA, NEELY & GRAHAM, P.C., P O BOX 1871, KNOXVILLE, TN, 37901, US

(21) Appl. No.: 11/934,274

(22) Filed: Nov. 2, 2007

(51) Int. Cl. A61B 5/00

(52) U.S. Cl. 600/306

(57) ABSTRACT

A system and method for documentation and analysis of dermatological aspects of a human body. The system has a predetermined arrangement of at least three image sensors selected from visible light sensors, ultraviolet (UV) light sensors, infrared (IR) sensors. Each of the image sensors has an effective normalized focal length of from about 8 to about 28 millimeters, an aperture stepped-down to at least f/4 or higher F-stop, and a shutter exposure length of no longer than about 125 milliseconds. Output from the image sensors provide a single relatively high resolution image of a skin surface of the human body obtained from a distance of at least about 0.1 meters. A data collection and processing system is integrated with the image sensors to provide storage, analysis, and output of in-situ dermatological information to a system operator.

### TECHNICAL FIELD

The disclosure relates to documentation and analysis of dermatological properties, and in particular to systems that provide improved capturing and analysis of images of skin surfaces for the purpose of aiding in the documentation, assessment, and treatment of the skin.

### BACKGROUND AND SUMMARY

Imaging portions of a body for documenting and tracking physiological and pathological changes over time has been useful for the purposes of early detection and treatment of a variety of conditions including cancer, burns, and the like. Visible light and multi-spectral cameras have been used to capture digital images of partial regions of the body. Typically, handheld cameras and scanner devices are used for the manual collection of images, usually by a skilled professional. Once collected, the images are manually inspected by a medical professional to determine the appropriate treatment regimen if any. Often, high resolution images of the body are limited to particular areas of interest and are not viewed in the anatomical context of an expansive total body image.

Additionally, in order to capture high resolution expansive imaging of the skin surfaces of the body, many sets of individual images are typically required that are then manually assembled into a full body image collection for individual image review. Such an imaging process is typically slow and requires positioning a subject in multiple predetermined positions, with multiple images being taken from each individual position. For example, a series of from about 30 to 60 images may be taken by a professional photographer over a period of 30 minutes to an hour and a-half per subject. The images are then compiled, printed in hardcopy or on a CD, and sent to a professional practitioner for future consultation with the subject.

Accordingly, what is needed is an integrated and automated system for imaging total visible skin areas that: captures total visible skin mages in about the time it takes to perform a chest x-ray or mammogram; provides images in a zoomable, interactive format; reduces the total number of images taken overall while increasing skin image detail viewable within the global context of the skin detail; accommodates both ambulatory and non-ambulatory subjects; may be configured to be portable; and provides analysis that aids in documentation, assessment and treatment of the skin.

In view of the foregoing, an exemplary embodiment of the disclosure provides a system for documentation and analysis of dermatological aspects of a body. The system has a predetermined arrangement of at least three image sensors selected from visible light sensors, ultraviolet (UV) light sensors, infrared (IR) sensors, and combinations of two or more of visible light, UV and IR sensors. Each of the image sensors has an effective normalized focal length of from about 8 to about 28 millimeters, an aperture stepped-down to at least f/4, and a shutter exposure length of no longer than about 125 milliseconds. Output from the image sensors provide a single relatively high resolution image of a skin surface of the body obtained from a distance of at least about 0.1 meters. The system optionally includes a geometric sensing component for providing three dimensional coordinate data corresponding to the imaged skin surface on one or more sides of the body. A data collection and processing system is integrated with the image sensors and optional geometric sensing component to provide storage, analysis, and output of in-situ dermatological information to a system operator.

In another exemplary embodiment there is provided a method for documenting and analyzing in-situ dermatological information. The method includes imaging a skin surface of a body from a distance of at least about 0.1 meters using a predetermined arrangement of at least three image sensors selected from visible light sensors, ultraviolet (UV) light sensors, infrared (IR) sensors, and combinations of two or more of visible light, UV and IR sensors. Each of the image sensors has an effective normalized focal length of from about 8 to about 28 millimeters, an aperture stepped-down to at least f/4 or higher F-stop, and a shutter exposure time of no longer than about 125 milliseconds to provide a single relatively high resolution image of the skin surface of the body. Geometric mapping data is optionally generated for the high resolution image to provide three dimensional coordinate data corresponding to the imaged skin surface. The image and optional mapping data is input to a data collection system that outputs in-situ dermatological information to provide high resolution interactive images.

Yet another embodiment of the disclosure provides a stand-alone skin surface imaging system. The system has a housing including a predetermined arrangement of at least three image sensors selected from visible light sensors, ultraviolet (UV) light sensors, infrared (IR) sensors, and combinations of two or more of visible light, UV and IR sensors. Each of the image sensors has an effective normalized focal length of from about 8 to about 28 millimeters, an aperture stepped-down to at least f/4 or higher F-stop, and a shutter exposure length of no longer than about 125 milliseconds. Output from the image sensors provide a single relatively high resolution image of a skin surface of the subject's body obtained from a distance of at least about 0.1 meters. An optional geometric sensing device selected from a photo-metric imaging device, a laser scanning device, a structured light system, and a coordinate measuring machine (CMM) may be included for providing three dimensional coordinate data corresponding to the imaged skin surface. A data collection and processing system is attached to the housing and is integrated with the image sensors and optional geometric sensing component to provide storage, analysis, and output of in-situ dermatological information to a system operator.

Exemplary embodiments of the disclosure may be used to capture high resolution total body digital photographs in a manner that is quick, automated, and consistent in quality due to reduction of human error. Accordingly, the disclosed embodiments may have applications across numerous medical areas of need as well as outside the field of medicine. For example, the systems and methods described herein may be suitable for non-invasive calculation of skin wound or burn size, shape, and depth, visualizations before and after cosmetic surgery, calculation of skin area affected by psoriasis or acne lesion counts, for following the effectiveness of certain drugs on skin disease, or for non-medical applications such as reverse engineering competitors products, ergonomic based design or made-to-fit apparel construction, among others.

Other advantages of the systems and methods described herein may be that the systems are readily scalable and adaptable to be used in a variety of locations and settings. The systems may be configured to be fixed or portable thereby providing more flexibility for use of the systems. Accordingly, the systems and methods may eliminate the need to have images produced by professional photographers remote from the physician or medical professional's office.

For the purposes of this disclosure, the term “effective normalized focal length” means image sensors sized to 35 millimeter film frame size (36 mm×24 mm), also known as a “full frame sensor”

### BRIEF DESCRIPTION OF THE DRAWINGS

Further advantages of the exemplary embodiments will become apparent by reference to the detailed description when considered in conjunction with the figures, which are not to scale, wherein like reference numbers indicate like elements through the several views, and wherein:

FIG. 1 is a schematic view of a skin documentation and analysis system according to the disclosure;

FIGS. 2A-2D are schematic representations of various planar arrangements of image sensors for a system according to the disclosure;

FIGS. 3A-3D are schematic representations of various multi-planar arrangements of image sensors for a system according to the disclosure;

FIG. 4 is a schematic representation of an image sensor according to the disclosure;

FIG. 5A is a perspective view of an image sensor device with a lens;

FIG. 5B is a frontal view of the image sensor device of FIG. 5A with the lens removed;

FIGS. 6A-6B are flow diagrams for sensor data processing according to the disclosure;

FIG. 7 is a flow diagram for an analytical procedure using image data records according to the disclosure;

FIG. 8 is a block diagram of a skin documentation and analysis system configured in a stand alone configuration;

FIG. 9 is a block diagram for a controller component of the skin documentation and analysis system of FIG. 8;

FIG. 10 is a block diagram for a skin documentation and analysis system configured for use with a local area network or wide area network configuration; and

FIG. 11 is flow diagram for operator input/office flow for a skin analysis system according to the disclosure.

### DETAILED DESCRIPTION OF EXEMPLARY EMBODIMENTS

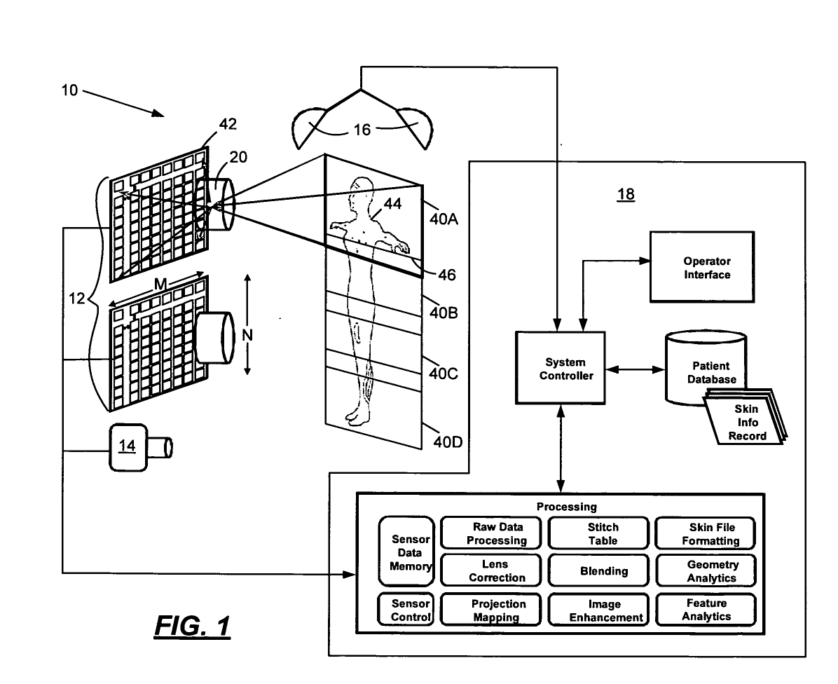

A schematic overview of a system according to an exemplary embodiment of the disclosure is illustrated in FIG. 1. The system 10 includes an imaging component 12, an optional geometry component 14, a lighting component 16, and a data processing component 18. The foregoing components are unified into a stand-alone system 10 that may be reconfigured or scaled to accommodate a variety of locations and purposes. Each of the components of the system 10 will be described in more detail below.

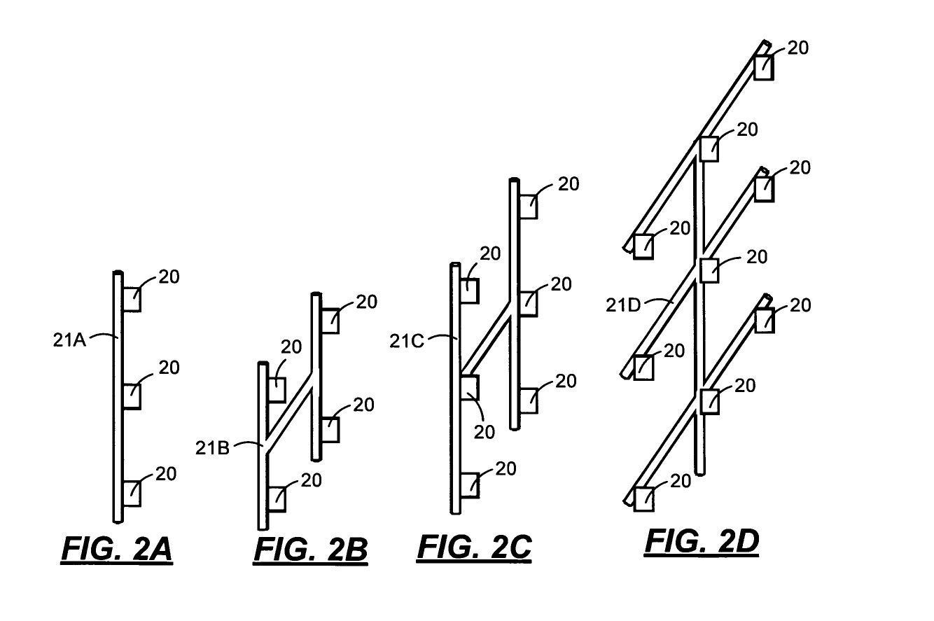

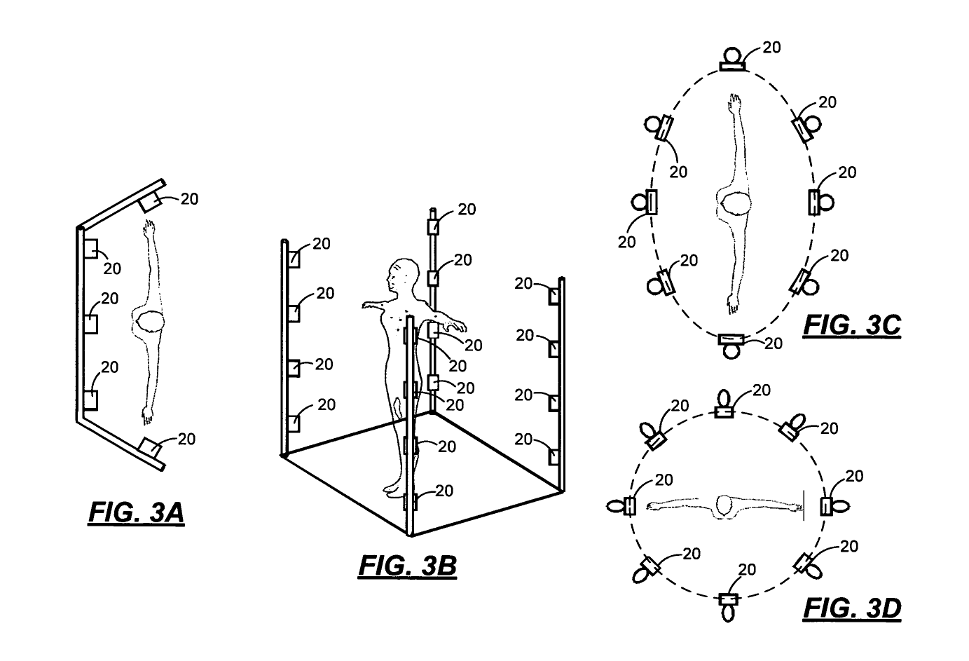

As shown in FIGS. 2 and 3, the imaging component 12 of the system 10 may be arranged in a variety of predetermined configurations. For the purposes of this disclosure, the imaging component 12 may include one or more image sensors 20 on support 21A (FIG. 2). At least three image sensors 20 are desirably used for the purposes of obtaining a full body image. The image sensors may be disposed in a single plane or in multiple planes as illustrated in FIGS. 1-3. In FIGS. 2A-2D, multiple image sensors 20 are arranged in planar configurations that may include a single linear arrangement of sensors 20 (FIG. 2A) or an x-y arrangement of image sensors 20 on supports 21B-21D as shown in FIGS. 2B-2D.

Multiple planar arrangements of image sensors 20 are shown in FIGS. 3A-3D. In FIG. 3A the image sensors 20 may be arranged in three separate planes in order to capture images on one side of the body. In FIG. 3B, the image sensors 20 are arranged in planes surrounding the body to capture images on all sides of the body. The multiple planes of image sensors 20 may be disposed in planes that define an arcuate arrangement of image sensors 20 as shown in plan view in FIGS. 3C and 3D. It will be appreciated that arcuate arranged image sensors 20 in FIGS. 3C-3D and the image sensors of FIG. 3A may be disposed in multiple planes along a vertical axis as indicated in FIG. 3B.

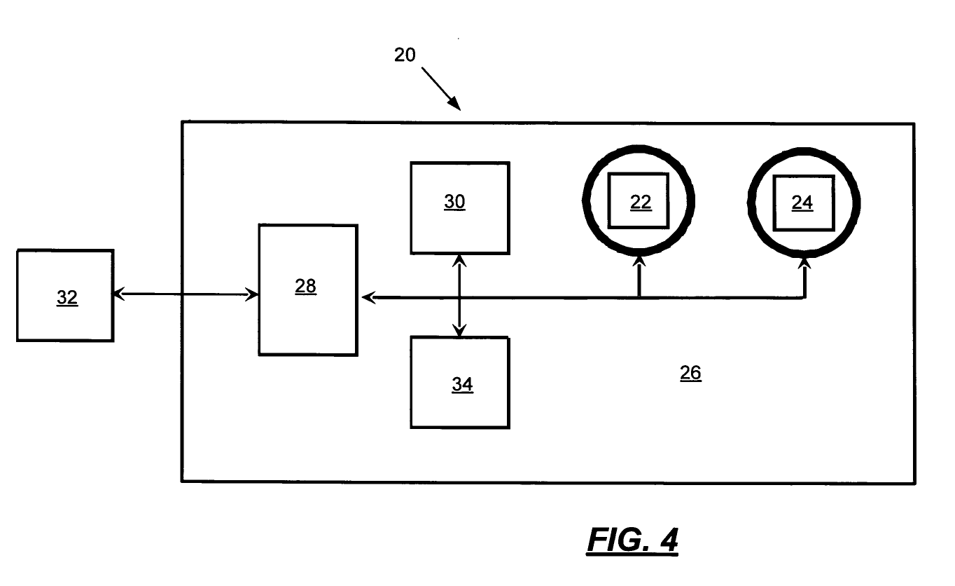

Each image sensor 20 may be a single visible light imaging component 22, or may include a combination of the visible light imaging component 22 and a second imaging component 24 selected from, a second visible light imaging component, an ultraviolet (UV) light sensing component, and an infrared (IR) sensing component as shown in FIG. 4. The imaging components 22 and 24 may be disposed on a circuit board or multiple connected circuit boards 26 that may include an input component 28, a sensor processing component 30, an output component 32, and a memory component 34.

### Image Sensors

Visible light imaging components 22 may be include a sensor chip that is used to detect electromagnetic spectrum (EMS) radiation reflected off the surface of skin. Sensor chips for commercially available visible light imaging components 22 typically convert reflected light into electrical voltages. A visible light imaging sensor chip is available from Micron Technology, Inc. of Boise, Id. The visible light imaging component 22, for example, is available from Lumenera Corporation of Ottawa, Canada, and includes a circuit board and a CMOS light sensor chip, processing, and input/output circuitry that can deliver from about three or more mega pixels of image resolution.

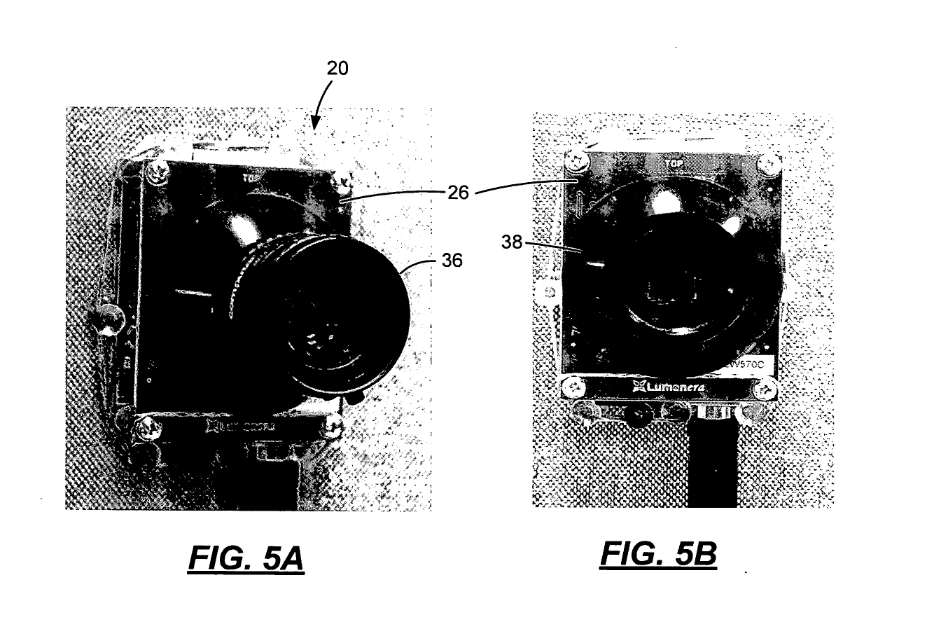

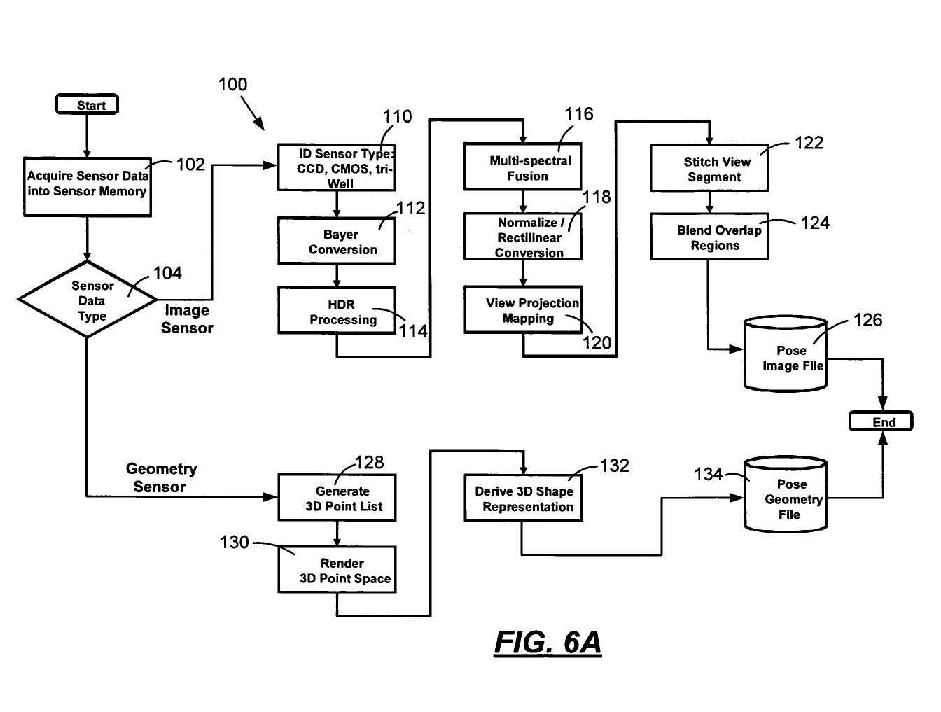

With reference to FIGS. 5A and 5B, each image sensor 20 may include a separate lens 36 to focus the reflected EMS radiation onto a sensor chip 38 associated with the sensor 20. Depending on the application, the lens 36 may be selected from a range of possible focal lengths. The focal length determines a field of view or size of a skin region imaged by the sensor 20. Also depending on the application, the lens 36 may be a fixed focus or variable focus lens, with either manual or programmatic control of the lens focus. A suitable focal length for purposes of the disclosure is an effective normalized focal length ranging from about 8 to about 28 millimeters. Each lens 36 has an aperture that is stepped-down to at least f/4 or higher F-stop and has a shutter exposure length of no longer than about 125 milliseconds under suitable lighting conditions.

Each image sensor 20 may be used to detect one or more spectral bands, including the ultraviolet light spectral band (0.001 μm to 0.3 μm wavelength), the visible light band (0.4 μm to 0.7 μm wavelength), and the infrared light band (0.75 μm to 1 mm wavelength). Other spectral bands shorter than 0.03 μm and longer than 1 mm may also be selected for inclusion in the image sensor 20.

With reference again to FIG. 1, each image sensor 20 captures a one or two dimensional grid 42 of samples corresponding to a skin surface regional field of view 40. The resolution of the image is defined as the total number of samples captured by an each sensor 20. For example, a two dimensional (2D) field of view has a planar arrangement having a grid width (M) and grid height (N). The 2D grid plane is also referred to as the sensor image plane. Resolution may vary according the requirements of the application. Each sensor 20 captures samples with a certain level of dynamic range that is characterized by a number of bits. For example, a 10-bit dynamic range sensor 20 may discriminate between 2¹⁰ or 1024 levels of intensity. Each sensor 20 may create a sample in an image memory that has a size equal to M*N*2¹⁰ bits. In the case of the one dimensional grid plane, the width may be 1.

The system 10 supports a full range of currently available image sensors 20. However, since the system 10 is composed of an open platform, currently available and future available sensor components 20 may be readily incorporated into the system 10. The system 10 is also configured to support future sensor designs that may result in the capture of an image data set of M*N*2^{O} pixels size, where M≧1, N≧1, and O≧1. Suitable minimum values for (M,N,O)=(1, 2048, 8). Sensor types that may be supported by the system include, but are not limited to:

CCD or CMOS linear sensors;

Tri-well linear sensors;

CCD or CMOS grid sensors;

Tri-well grid sensors;

Micro-cantilever sensors;

SKINCHIP Sensors or variations based on designs developed for biometric fingerprint recognition;

Parallel optical axis sensors; and

High dynamic range sensors.

Two or more sensors 20 may be spatially configured to sample adjacent field of view regions 40A-40D of a skin surface 44. Sensors 20 may be placed so that the regions sampled on the skin are abutted at an edge 46. Alternatively, regions sampled may overlap across one or more sensors 20. The multiplicity of sensors 20 operates in parallel to capture a higher level of detail and resolution than would be possible through the use of a single sensor 20. For example, as shown in FIG. 1, sensors 20 may be used to simultaneously capture four slightly overlapping field of view regions 40A-40D.

Sensor grid image planes 42 may be arranged in various relative spatial configurations and quantities based upon the requirements of the application. In one application, all sensor grid image planes 42 share a common plane as shown in FIGS. 2A-2D. FIGS. 2A-2D illustrate arrangements of sensors 20 in homogeneous, aligned, row-by-column configurations. FIGS. 3A-3D illustrate arrangements of sensors 20 in non-homogeneous, non-aligned configurations such as spherical, cubic, or cylindrical orientations of the sensor grid image planes 42.

Sensors 20 may be placed in a landscape or portrait orientation, or in mixed landscape and portrait orientations. In either case, the arrangement is designed to capture in ultra-high resolution those details that are needed for the application. Spacing between the sensors 20 may be based on the field of view sampled, the lens parameters, and the working distance between the sensor image plane 42 and the object 44 being imaged. The number of sensors 20 used in the system 10 may be based on the desired resolution and on how many or how few poses may be required in order to capture the total skin area desired.

### Geometry Sensors

With reference again to FIG. 1, geometry components 14 may be used to capture the size and shape of the object 44 being imaged. The geometry components 14 generate three dimensional (3D) coordinate data that corresponds to the skin surface detail. The resolution of the geometry component 14 may vary according to the application and the component selected to provide the geometry data. Typical sampling rates may be in the 10s of thousands of points per object orientation.

Because the system 10 is composed of an open platform, currently available and future available geometry components 14 may be incorporated into the system 10. Accordingly, embodiments of the system 10 may use photo-metric or stereo imaging, laser scanning, or a structured light system as the geometry component 14. In each case, a point cloud of 3D data is generated that corresponds to the skin surface geometry. Other sensors such as a coordinate measuring machine (CMM) may be used, albeit with slower acquisition time because of sequential point collection.

3D point cloud data may be processed to provide a more flexible representation for file storage and analytics. Conversion to NURBS or polygon mesh format is well known, and provides an optimization for storage requirements and processing flexibility.

A number of techniques for geometric sensing are based on image-based modeling techniques that rely on photogrammetric calculations. Use of stereo-based triangulation is a very well known technique that allows for calculation of size and geometry of areas of interest. Other approaches use active sensing techniques based on eye-safe lasers or other reflective modalities of capture to determine size and geometry of areas of interest. The coordinate measurement machines manually trace key geometries of the body, allowing a coarser level geometric capture than may be possible using laser or structured light techniques.

While various existing approaches to the collection of skin-related geometry data provide potentially adequate levels of spatial detail, these systems alone do not offer seamless integration with single or multi-spectral light collection devices, not with analytics that may be useful for identifying regions of interest or for performing automatic calculations of key feature size and shape.

### Lighting

The lighting component of the system 16 provides for the illumination of skin regions being sampled by the sensors 20. Lighting will provide for reflected light illumination of the subject's skin. Depending on the sensor array configuration (planar, arcuate, etc.), the selected sensor lens focal length, F-stop, and sensor array working distance to the subject, the lighting will be placed so as to illuminate all areas that are desired to be captured by the sensors.

In general, located proximate to a single sensor 20, or a subset of closely spaced sensors, will be a single lighting source. A typical minimum system will be configured with at least two light sources to ensure full illumination of a particular pose with a wattage and/or Lux level set based on the sensor array configuration, selected sensor lens focal length, F-stop, and sensor array working distance to the subject.

Lighting may be either ambient or strobe. Ambient lighting will continuously illuminate the subject's skin allowing for a flexible duration of digital sensor exposure and image readout. This lighting will be most appropriate for a CMOS sensor chip 22 or other current or future sensor designs in which pixels in each image frame are sequentially exposed via a rolling shutter. Strobe lighting provides non-continuous illumination of the subject's skin. The lighting is activated synchronously with the digital sensor exposure and image readout. Strobe lighting may be appropriate for a CCD sensor chip 22 or other current or future sensor designs in which pixels in each image frame are simultaneously exposed via a global shutter. Strobe lighting may take advantage of sensor output signaling that occurs during exposure, allowing the strobe light to fire at the appropriate time. Use of ambient or strobe lighting may be implemented with either CMOS, CCD, or other current or future based sensor chips 22. The foregoing criteria assume that the light density per surface area (or Lux) of the light source and the sensor exposure and image readout rates are low enough to ensure for a full image readout from the sensor, given the particular lens aperture.

The light source may be varied, and will depend on the types of image sensors included in the system. For visible light sensing, the light source may be either incandescent (tungsten, neodymium, halogen), fluorescent (T8 or T12), metal halide, xenon, mercury vapor, high and low pressure sodium, or other type of visible light component. Light sources may also include ultraviolet (UV) lighting.

Light sources may be diffused, allowing for a softening of the combination of light sources to provide overall illumination of the subject's skin. Diffusion may be achieved through several approaches, including use of a softbox that uses translucent white diffusing fabric or reflectors that bounce the light off a secondary surface to scatter the light.

Suitable illuminations may correspond to the EMS band or bands to which the sensors 20 are tuned. Suitable lighting will be incandescent lamps having wattages ranging from about 250 watts to about 1000 watts. Lighting parameters may be calibrated at system startup to ensure system color and white balancing of 18% grey, ensuring consistency across skin images.

### Skin Data Processing

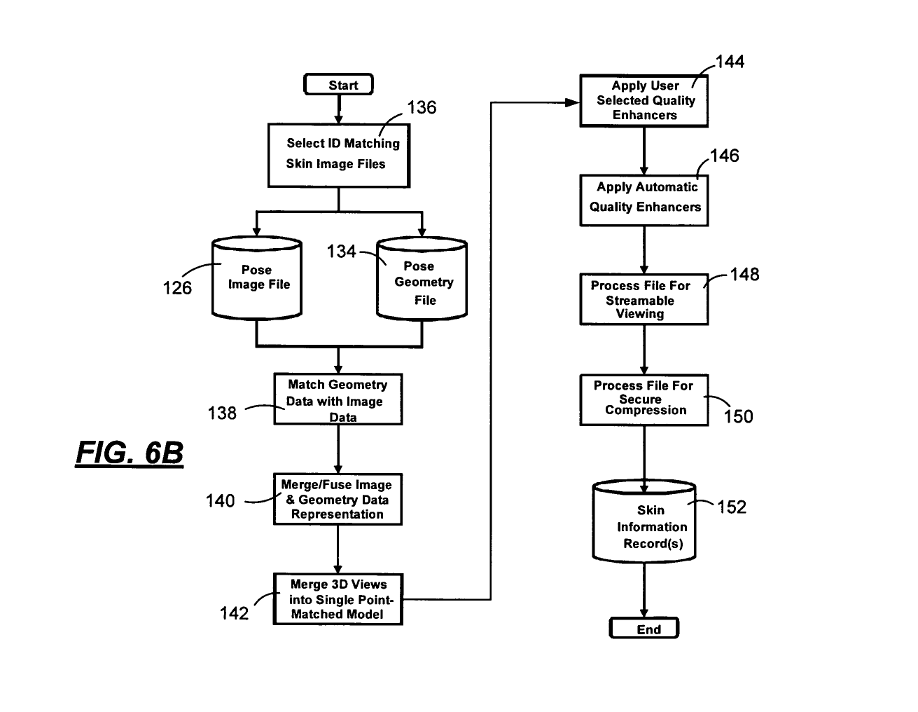

Skin data for each subject may be processed according to a process flow diagram 100 illustrated in FIGS. 6A-6B. The process is dependent on the type sensor 20, number of sensors 20 in the system, arrangement of sensors 20, whether the sensors 20 are moved across the object or fixed, and whether the object is moved or fixed. Image processing that is supported by the system 10 may include, but is not limited to:

Bayer pattern processing;

Dynamic range processing;

Registration and fusion of images taken in two or more EMS spectral bands;

Correction for parallax error;

Correction for curvilinear distortion (barrel or pincushion);

Interframe point matching;

Interframe image stitching; and

Overlap image area blending.

Commercially available software programs that may be used to provide the foregoing processing include, but are not limited to, a LUMENERA USB Camera API (LuCam API) software developer kit. Certain aspects of these processing steps may also be implements using proprietary algorithms.

With reference to FIG. 6A, in a first step 102 of the process, data is acquired by the image component 12 and geometry component 14 and is routed in step 104 to an image processing element 106 or to a geometry processing element 108 based on the type of data acquired in step 102. In the image processing element 106, the image sensor type is selected in step 110, converted in step 112 to data bits that are processed in step 114. Individual images are combined into a single super high resolution total body image using an image stitching algorithm. The system 10 may be adaptable to use a variety image stitching techniques. The output from step 114 is input to step 116 to provide multi-spectral fusion of the image. Accordingly, image processing includes registration of images from sensors 20 for the creation of a single multi-spectral image. From there, the image is normalized in step 118, and mapped to a projected image in step 120. Individual segments are stitched together in step 122 and overlapped regions, if any, are blended together in step 124 to provide an image file 126 for each pose. Image based modeling is used to generate two dimensional or three dimensional perspectives. Commercially available software programs that may be used to provide the foregoing processing include, but are not limited to, Eos Systems Inc. PHOTOMODELER or the Realviz S. A. STITCHER and IMAGEMODELER products. Certain aspects of these processing steps may also be implemented using proprietary algorithms.

In the geometry processing element 108, the data from the geometry component 14 is collected to provide a 3D point list in step 128 that is used to render a 3D point space in step 130. From the point space, a 3D shape representation is provided in step 132. The shape representation is then saved in a geometry file 134 for each pose. Commercially available software programs that may be used to provide the foregoing geometric processing include, but are not limited to, GEOMAGIC STUDIO 9 from Geomagics. Certain aspects of these processing steps may also be implemented using proprietary algorithms.

Once the pose files for the image and geometry data are compiled for a given subject, that information may be used to provide a skin information record that can be used to assess changes in skin properties or characteristics. The flow diagram for creating the skin information record from the image and geometry pose files is given in the flow diagram of FIG. 6B. Access to a skin file for a subject is provided by inputting a unique ID in step 136. The ID is used to select the corresponding image file 126 and geometry file 134 for that ID. The geometry data and image data from the files 126 and 134 are matched in step 138 and merged together in step 140 to provide merged 3D views in step 142. Image quality enhancers may be applied manually in step 144 and automatically in step 146. The file is then processed for streamable viewing in step 148 and is compressed for secure storage in step 150 to provide the skin information records 152.

In an alternative embodiment, in addition to mapping the skin onto the actual geometry captured and processed directly from the subject in order to provide the 3D views in step 142, a 3D view may be provided by mapping the skin image onto a stylized 3D model representation of the subject. In this case, an existing selection of predefined 3D body geometry models may be used. A closest match model may be digitally modified to match key measurements of the subject's skin (e.g., height, waist and chest circumference, arm length, etc.). The skin data is then mapped onto this representative model to facilitate a more natural interactive 3D viewing of the total visible skin image data.

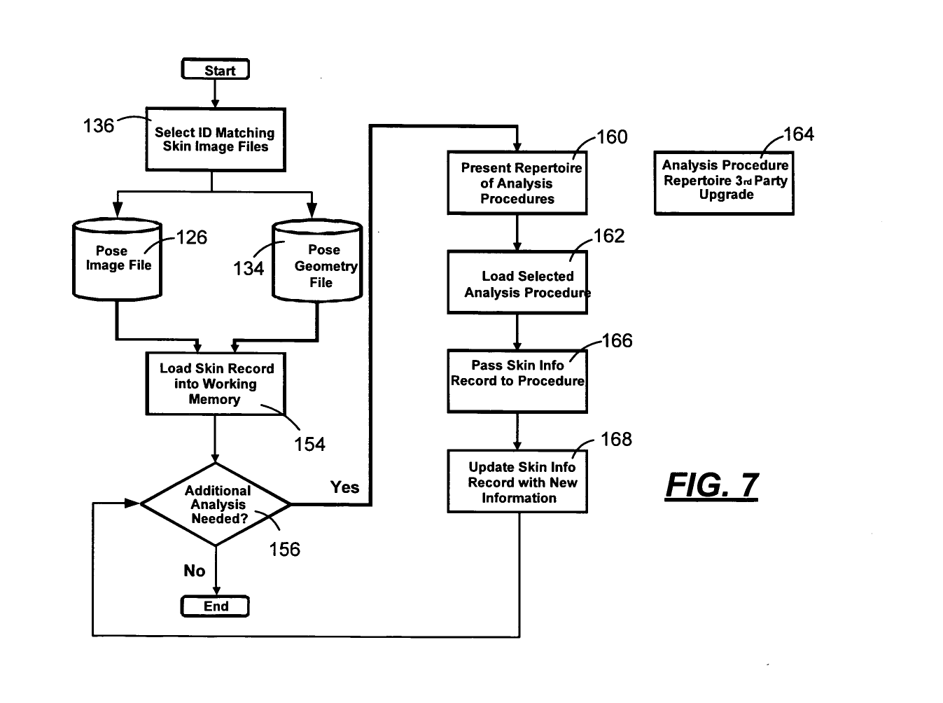

Once the records 152 are created, they may be used to determine changes in skin properties or characteristics according to the analytical procedure shown in the flow diagram of FIG. 7. The process includes inputting a unique ID of the subject in step 136 to access the image file 126 and geometry file 134 that is used to access the skin records 152 (FIG. 6B). The skin records 152 are input into a working memory location in step 154. From the working memory location, a determination is made in step 156 whether or not to perform analytical procedures on any one or more portions of the image. If analytical procedures are required, the data from the memory location in step 154 is input into a predetermined set of analytical procedures in step 160. Additional procedures may be input in step 162 to complement the procedures included in step 160. Likewise, the system is adaptable to including upgrades 164 of analytical procedures from third parties. The skin information is analyzed by the analytical procedure in step 166 and the skin information record 152 is updated in step 168 with the analytical analysis provided in step 166. The skin data is run through a variety of mathematical and cognitive based routines to derive direct and indirect data about the skin. The data is then contexted and added as additional information to the skin record. The analysis step utilizes a prior knowledge base of skin information and models of skin analytics based on actual experience. Analysis of the prior knowledge against the new skin information enables decisions to be made about the current skin information and to assess levels of confidence related to inferences made during the analysis.

For each feature identified on the skin for analysis by the system 10, the system may perform shape determination, length, width, depth, area, volume, percentage of total visible skin, number of lesions, as well as color, brightness, saturation, edge contrast measurements, and the like. Feature measurements may be performed automatically or interactively using a ‘ruler overlay’ graphic or other interactively placed measurement marker graphic over the feature of interest. The feature may be identified by comparison to a database of feature properties or by use of a neural network, Bayesian statistical, or other computational algorithm to identify the feature. The system generates sizes of key skin and/or body features, either as a predefined series of measurements reported automatically, or calculated interactively with operator input. Image data is normalized for each subject so that comparison of images taken at different times of the same subject can be overlaid for image processing (i.e. image ‘subtraction’ to identify changes, or size calculations to determine growth; comparisons of measured areas, and the like). Commercially available software programs that may be used to provide the foregoing processing include, but are not limited to, ITT's Visual Information Solution product, IAS Image Access Solutions or the Tina open source project for medical image analysis.

The system 10 may include a keyboard, and/or touch sensitive screen for all system setup, operations, and maintenance and for inputting commands for imaging and analysis procedures. Operator inputs may include, but are not limited to, creating new skin image records for a subject, capture one or more poses, access for comparing previous subject's skin image records, selecting skin image feature analysis to be performed, and output of a variety of preformatted or custom layouts of the skin image record data. The operator may also be able to select system preferences, recalibrate the system, initialize the system for staring an image capture operation, annotate skin information records with text, graphical icons, or freehand graphical edits, perform interactive image processing on skin information record data to enhance the record data, such as contrast, brightness, saturation, etc., display an interactive magnifying glass to view details of the skin image record in high-level magnification, and select same pose images captured at different times in order to perform a comparative analysis.

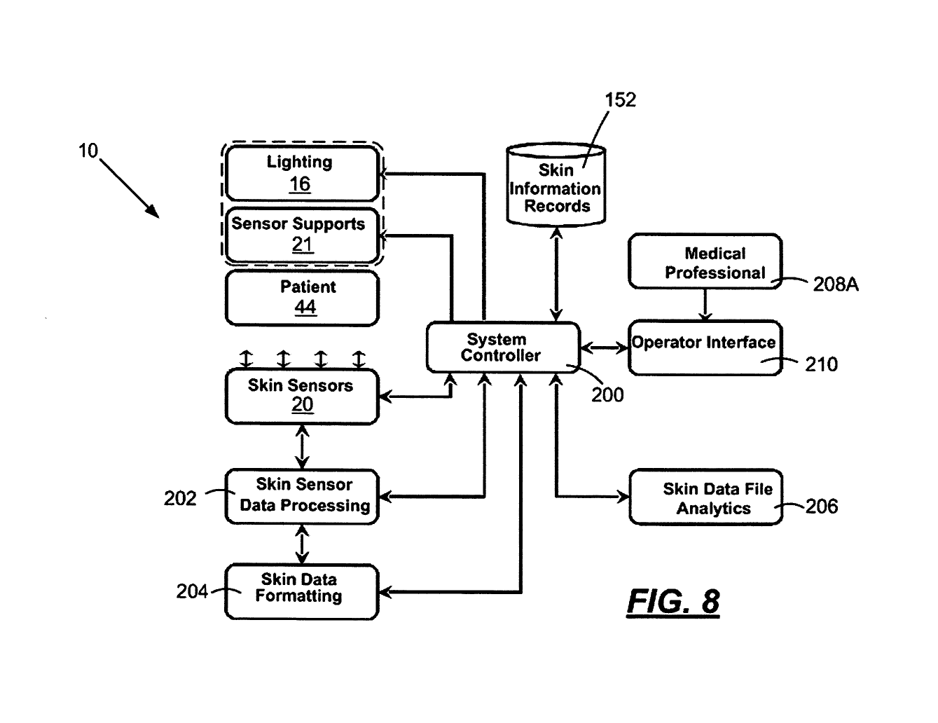

FIG. 8 provides a functional illustration of the system 10 and the interaction of the various components thereof. As shown in FIG. 8, a system controller 200 provides control of the lighting 16, and in the case of movable sensors 16, also to the sensor supports 21 as they scan over a subject 44. Sensor supports 21 also may include physical and visual aids to enable the subject to be positioned for pose image capture. The system controller 200 also includes input and output from a skin sensor processor 202 that provides output to the sensors 20 for controlling the imaging process and the data collection process. Data from the skin sensors 20 is formatted in a skin sensor data formatting unit 204 before it is input to the system controller 200. Previous records or stored skin data records from the skin information records component 152 may be input to the system controller 200 for comparison purpose or new image date may be stored in the records component 152. The system controller 200 also provides input and output to a skin data file analytics component 206. Individual assessment of skin data may be provided by a medical professional 208A by means of an operator interface 210 to the system controller 200.

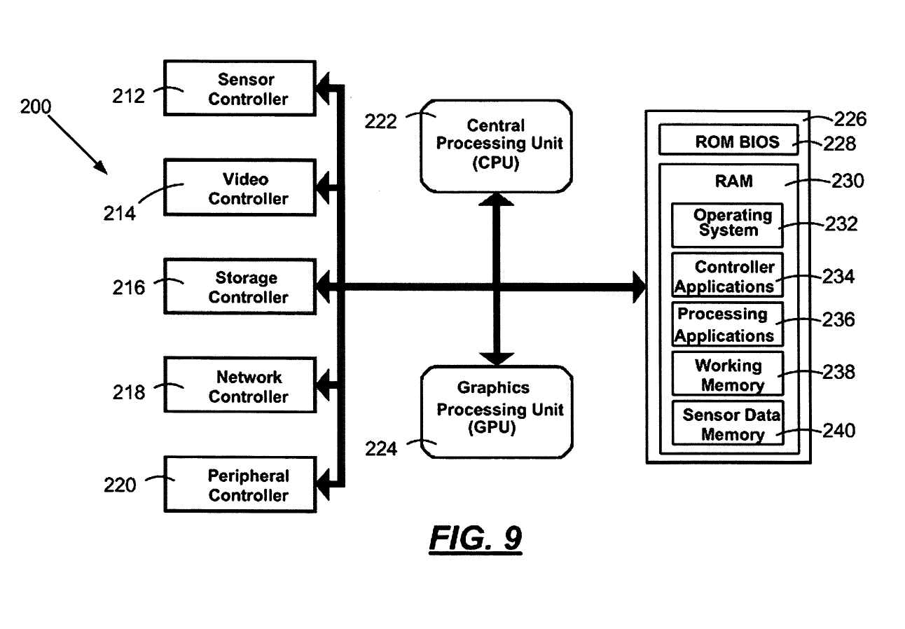

Components of the system controller 200 are illustrated in FIG. 9. The system controller 200 may include a sensor controller 212, for controlling input of visible, infrared, ultraviolet, or other EMS bands from the sensors 20 and for geometric data input from the geometry components 14. A video controller 214 may be included in the controller 20 for use with a display or touch screen input system. A storage controller 216 provides access to mass storage of data by use of optical, magnetic, or other storage means. Data storage may be provided on a CD or DVD, or may be included in a fixed or removable hard drive unit. If the system is interconnected to a network for remote access of the data and images, a network controller 218 may also be included. The network controller provides access via a LAN, WAN or ETHERNET system. Access may also be provided by a wireless system such as, BLUETOOTH, ZIGBEE, ultra wide band or other wireless systems. A peripheral controller 220 may be included for use with a mouse, keyboard, printer, universal serial bus (USB), or other input devices.

The controllers 212-220 described above are controlled by a central processing unit 222 and optional graphics processing unit 224. The controllers 212-220 are also in processing communication with a memory component 226 that includes ROM memory 228 for the system bios and RAM memory 230 for the operating system, 232, controller applications 234, processing application 236, working memory 238, and sensor data memory 240.

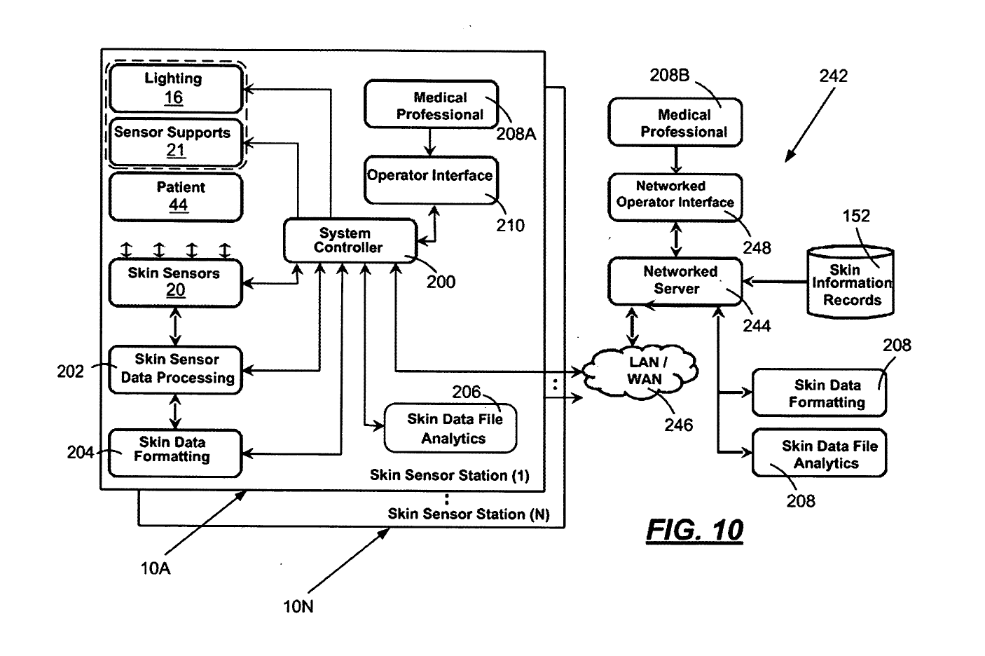

FIG. 10 illustrates a system 242 for remote access of the data generated by each of the systems 10A to 10N, wherein N is the number of systems networked together. In this system, the skin information records 152 are stored on a network server 244. The network service 244 is in communication with each of the systems 10A-10N through a LAN or WAN network 246. The network server 244 also includes a networked operator interface 248 for access by a medical professions 208B that may be the same or different from the medical professional 208A. The medical professional 208 B may have access to a skin data formatting component 250 and skin data file analytics 252 for providing a treatment plan.

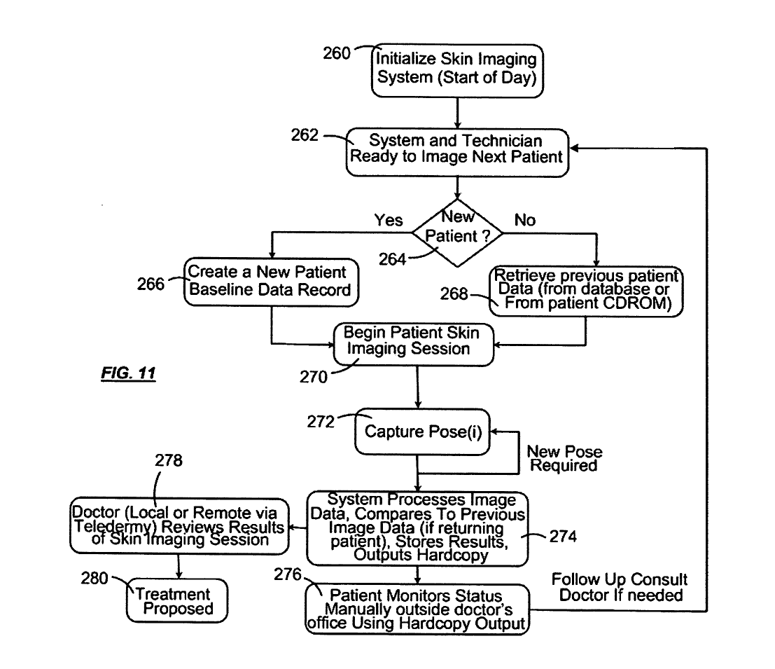

A flow diagram for an operator or technician using the system 10 is illustrated in FIG. 11. An operator initializes the system 10 in step 260 so that the system is ready in step 262 for a new image collection. In step 264, a determination is made by the operator if the image is for a new record or for updating data from a previous record. If the data is for a new record, a new record is created in step 266. If the data is for an existing record, the previous data record is retrieved in step 268. In the next step 270, an imaging session is begun. The poses of the subject are captured in step 272 until all poses are captured. The system then processes the image data and compares the new image data to previous image data in step 274 according to the processes described above. At this point, the subject may be given a hard copy of the image data analysis in step 276. In the alternative, the data and comparison from step 274 may be sent to a remote location for review by a professional in step 278. If necessary, the professional may provide a treatment plan in step 280 for any conditions identified that need treatment.

As described above, the system 10 enables collection and analysis of skin data in an integrated system that can be readily changed or reconfigured to include more or fewer components. For example, the system 10 may include a component for automatic identification, assessment, and classification of lesions and moles on a subject. Specialized algorithms may be used in the system for identifying the shape, color, and size of lesions or moles and comparing the analytical results of the image records to previously cataloged images for comparative purposes. Since the high resolution images capture the entire skin surface in situ, there is no need to image only select areas of the skin. The system 10 may be adapted to not only identify melanomas, but may include components to identify other skin diseases or skin changes over time. Accordingly, a single system 10 may be configured to handle multiple skin features including, but not limited to, lesions, melanomas, wound size and shape, aging, burns, psoriasis, acne, forensic data, and the like. The system 10 may also be used for skin cancer screening, burn treatment, plastic surgery, endocrinology, medical education, drug interaction studies, forensic, trauma, and cosmetics.

One important advantage of the system 10 described herein is that the imaging, comparisons and analysis may be performed in a single session in a single location. The images may also be transmitted electronically to a remote location by the system 10 for consultation or further treatment recommendations without the subject having to travel or be transported to the remote location. Since the system 10 automatically captures, catalogs and stores the image data, there is very little time lag between image capture and analysis. The total number of photographic images in high resolution that may be needed to capture the entire skin area of a human body may be 1 to 8 photographs in contrast to the 30 to 60 photographs required by conventional skin imaging systems. Accordingly, imaging the entire body may take about the same amount of time as obtaining a chest x-ray.

The system 10 may be automated for capture and analysis of the images so that systems may be mobile or may be located in multiple locations rather than a central location. Because the image collection and analysis are integrated into a single system, the system 10 may be used without the need for a professional photographer. Image records may be provided electronically as well as in hard copy or on a CDROM if desired.

A particularly useful application of the system 10 is for the identification and treatment of skin cancer. Skin cancer may be of several different types, but the most deadly form is malignant melanoma. Such cancer usually starts with the appearance of a mole, at first perhaps benign in appearance, but one that changes over time. Change often occurs on the outer surface layer of the skin (epidermis) in which the mole may broaden and take on more ominous characteristics. Over time the melanoma may begin to spread to the underlying skin layers increasing the risk of metastasis. Additionally, individuals with dysplastic nevi syndrome who have hundreds or over a thousand moles on their body surface must be frequently and carefully examined due to the atypical and uncertain nature, benign vs. cancerous, of these moles. It is often unreasonable and unethical to remove all the suspicious atypical moles on such individuals when many of these moles are typically benign.

Accordingly, the use of the system 10 described herein for skin cancer screening, particularly those at high risk for malignant melanoma or atypcial dysplastic nevi syndrome is invaluable. Software used in the system can analyze the total body skin images (one or serial images) for mole characteristics suspicious for melanoma and a report may be automatically generated for use by a professional.

Likewise, the system 10 has application for the documentation, analysis and treatment of burns on the skin. It is important to accurately documents burns in order to provide the most effective treatment plan. The system including specialized analytical software may be used to classify burns and the percentage of the skin that is burned. The system may also be used to document the healing process through serial photographs, particularly in the case of skin grafts.

In the case of skin trauma, the system 10 may be used to document and identify damages to the epidermis and any underlying layers (dermis and subcutaneous tissue) that may be exposed. Particularly in the case of trauma to the skin, the system may be configured to image a person's skin while the subject is lying down.

Plastic surgery continues to expand from alteration or repair of smaller areas such as nose or confined regions such as in breast augmentation to now larger and multiple areas of the body. An example of such plastic surgery may be multiple large regions of the body going thru skin resection related to significant weight loss. Accordingly, the system 10 may provide documentation of large global regions of the skin before and after surgical intervention.

Other applications of the system 10 may include the use of the system for endocrinology in which body habitus, development and maturation may be more adequately documented over time. The system 10 may also be used for research and study of skin changes that occur with aging. Other configurations of the system may be used for cosmetic documentation and analysis regarding skin tone and damage. Additionally, the system 10 may be used in the fields of medical education, as well as in forensics. While the description and figures are particularly directed to imaging human skin, the system 10 is not limited to such applications. Accordingly, the system 10 may be adapted to veterinary medicine uses such as imaging both large and small animals.

The foregoing embodiments are susceptible to considerable variation in its practice. Accordingly, the embodiments are not intended to be limited to the specific exemplifications set forth hereinabove. Rather, the foregoing embodiments are within the spirit and scope of the appended claims, including the equivalents thereof available as a matter of law.

The patentees do not intend to dedicate any disclosed embodiments to the public, and to the extent any disclosed modifications or alterations may not literally fall within the scope of the claims, they are considered to be part hereof under the doctrine of equivalents.

What is claimed is:

1. A system for documentation and analysis of dermatological aspects of a human body, the system comprising:
a predetermined arrangement of at least three image sensors selected from the group consisting of visible light sensors, ultraviolet (UV) light sensors, infrared (IR) sensors, and combinations of two or more of visible light, UV and IR sensors, each of the image sensors having an effective normalized focal length of from about 8 to about 28 millimeters, an aperture stepped-down to at least f/4 or higher F-stop, and a shutter exposure length of no longer than about 125 milliseconds, wherein output from the image sensors provide a single relatively high resolution image of a skin surface of the human body obtained from a distance of at least about 0.1 meters;
optionally, a geometric sensing component for providing three dimensional coordinate data corresponding to the imaged skin surface on one or more sides of the human body; and
a data collection and processing system integrated with the image sensors and optional geometric sensing component to provide storage, analysis, and output of in-situ dermatological information to a system operator.

2. The system of claim 1, wherein the predetermined arrangement of image sensors comprise at least one linear array of image sensors.

3. The system of claim 1, wherein the predetermined arrangement of image sensors comprise at least one x-y array of image sensors.

4. The system of claim 1, wherein the predetermined arrangement of image sensors comprise multiple arrays of image sensors configured to capture a single relatively high resolution image of at least one side of the human body.

5. The system of claim 4, wherein the multiple arrays of image sensors are configured to provide a single relatively high resolution image of all sides of the human body.

6. The system of claim 1, wherein the predetermined arrangement of image sensors comprise an array of at least three cameras having megapixel resolution wherein the cameras are synchronized to provide the single, relatively high resolution image.

7. The system of claim 1, wherein the geometric sensing component comprises a device selected from the group consisting of a photo-metric imaging device, a laser scanning device, a structured light system, and a coordinate measuring machine (CMM) to provide a single dimensional data set for the one or more sides of the human body.

8. The system of claim 1, wherein the predetermined arrangement of image sensors are disposed within a single plane.

9. The system of claim 1, wherein the predetermined arrangement of image sensors are disposed in two or more planes.

10. The system of claim 1, wherein the predetermined arrangement of image sensors are disposed within an arcuate surface.

11. A mobile imaging unit comprising the system of claim 1.

12. A method for documenting and analyzing in-situ dermatological information, comprising:
imaging a skin surface of a human body from a distance of at least about 0.1 meters using a predetermined arrangement of at least three image sensors selected from the group consisting of visible light sensors, ultraviolet (UV) light sensors, infrared (IR) sensors, and combinations of two or more of visible light, UV and IR sensors, wherein each of the image sensors having an effective normalized focal length of from about 8 to about 28 millimeters an aperture stepped-down to at least f/4 or higher F-stop, and a shutter exposure length of no longer than about 125 milliseconds, to provide a single relatively high resolution image of the skin surface of the human body;
optionally, generating geometric mapping data for the high resolution image to provide three dimensional coordinate data corresponding to the imaged skin surface; and
inputting the image and optional mapping data to a data collection system; and
outputting the in-situ dermatological information to provide high resolution interactive images.

13. The method of claim 12, wherein the relatively high resolution image is provided by a linear array of image sensors, wherein the imaging step comprises scanning the skin surface as the image sensors and body move relative to one another.

14. The method of claim 12, wherein the relatively high resolution image is provided by an x-y array of image sensors.

15. The method of claim 12, wherein the relatively high resolution image is provided by a three dimensional array of image sensors.

16. The method of claim 12, wherein the geometric mapping data is generated using a device selected from the group consisting of a photo-metric imaging device, a laser scanning device, a structured light system, and a coordinate measuring machine (CMM).

17. The method of claim 12, further comprising categorizing changes in skin surface features over time.

18. The method of claim 15, further comprising identifying dematological aspects of the subject's body using heuristic knowledge-based processing techniques.

19. The method of claim 15, further comprising identifying dematological aspects of the subject's body using statistical-based processing techniques.

20. A stand-alone skin surface imaging system, comprising:
a housing including:
a predetermined arrangement of at least three image sensors selected from the group consisting of visible light sensors, ultraviolet (UV) light sensors, infrared (IR) sensors, and combinations of two or more of visible light, UV and IR sensors, each of the image sensors having an effective normalized focal length of from about 8 to about 28 millimeters, an aperture stepped-down to at least f/4 or higher F-stop, and a shutter exposure length of no longer than about 125 milliseconds, wherein output from the image sensors provide a single relatively high resolution image of a skin surface of the human body obtained from a distance of at least about 0.1 meters;
an optional geometric sensing device selected from the group consisting of a photo-metric imaging device, a laser scanning device, a structured light system, and a coordinate measuring machine (CMM) for providing three dimensional coordinate data corresponding to the imaged skin surface; and
a data collection and processing system attached to the housing and integrated with the image sensors and optional geometric sensing component to provide storage, analysis, and output of in-situ dermatological information to a system operator.

21. The system of claim 20, wherein the predetermined arrangement of image sensors comprise at least about three cameras having megapixel resolution wherein the cameras are synchronized to provide the single image relatively high resolution image.

22. The system of claim 21, wherein the predetermined arrangement of image sensors are disposed in two or more planes.

23. The system of claim 20, wherein the image sensors comprise visible light sensors further comprising an illumination system disposed in the housing.
